(19) United States
(12) Patent Application Publication
(10) Pub. No.: US 2005/0245791 A1
(43) Pub. Date: Nov. 3, 2005

(54) Shockwave method and apparatus for the application on the body

(76) Inventor: Wolfgang Bauermeister, Munchen (DE)

Correspondence Address: PARKHURST & WENDEL, L.L.P., 1421 PRINCE STREET, SUITE 210, ALEXANDRIA, VA, 22314-2805, US

(21) Appl. No.: 10/834,969

(22) Filed: Apr. 30, 2004

(51) Int. Cl. A61B 5/05

(52) U.S. Cl. 600/300; 600/407

(57) ABSTRACT

Diagnosis or treatment of myofascial trigger points and/or other tissue using focused or radial shockwaves. A shockwave emitter for such use is disclosed also.

The invention relates to the use of shockwaves in the field of medicine and in particular to the use of shockwaves in the field of Orthopaedic and Physical Medicine & Rehabilitation.

It has been known for quite some time that shockwaves which were initially used for the disintegration of kidney stones could be utilised in the field of Orthopaedic and Physical Medicine & Rehabilitation for pain relief, see Ref.: DE 43 03 830.1, DE 4341323.4 (Ref.: U.S. Pat. No. 5,727,556).

It is known that radial shockwaves can be utilized in the field of Orthopedic and Physical Medicine & Rehabilitation as published in “Indikations-und Handhabungsanleitung” of the company EMS(=Electro Medical Systems), Ch. de la Vuarpillière 31, CH-1260 Nyon, for the device Swiss DolorClast®”.

From the “Ergänzung ((supplement)) zur Gebrauchsanleitung FG172/2” (“Swiss DolorClast®/TST-Trigger-Stoβwellen-Therapie/Gebrauchsanleitung/Ergänzung zur Gebrauchsanleitung FB 172/2”), named “FB229/2 Version D/01/03” it is known that the radial shockwaves of the above mentioned device can be used for the treatment of Myofascial Trigger Points (MTrPs). The inventor of the present application broadened the spectrum of indications of the above mentioned device “Swiss DolorClast®” for the treatment of MTrPs which cause the Myofascial Pain Syndrome (MPS). The terms used are explained in the mentioned “Ergänzung zur Gebrauchsanleitung FG172/2” and it is included by reference into this application.

With the device of EMS a depth of penetration (measured with hydrophone) at −6 dB (Dezibel) of 2.7 mm for the applicator 6 mm (diameter) and of 8.0 mm for the applicator 15 mm (diameter) is achieved.

It is an object of the invention is to still improve the efficacy of the therapy of MTrPs and other tissues.

This can be achieved according to the invention by utilizing focused shockwaves for the treatment of MTrPs.

Focused shockwaves may, according to the invention, also be applied to other body tissues with considerable advantage.

According to the invention in a preferred method according to the invention the focused shockwaves have a Total Energy Flux Density (ED) between 0.10 mJ/mm² and 3.0 mJ/mm.

The total energy flux density is the sum of the positive and the negative energy flux density.

The use of focused shockwaves in the field of Orthopedic and Physical Medicine & Rehabilitation is generally known i.e. through the device Piezoson 100, of the company Richard Wolf GmbH, D-75438 Knittlingen, Pforzheimer Straβe 32. In the instruction manual which is available in English as well GA-A 174/en/Index: 01-03-4.0/ÄM: KG 03-009 and which is referred to explicitly herein, it is stated in detail how the focusing of the shockwave is achieved through the spherical orientation of several Piezo-Elements.

However, up until this invention nobody proposed to utilize focused shockwaves also for the treatment of MTrPs. Still, the use of focused shockwaves has considerable advantages for the treatment of MTrPs. The Trigger areas can be addressed more precisely and can be reached and treated even in deep lying body structures.

In the above mentioned “Ergänzung zur Gebrauchsanleitung FB 172/2, FB 229/2 Version D/01/03”, it is stated (point 1.2 on page. 4), that it is necessary to locate the MTrP precisely in order to achieve a successful causal therapy. The localization of the MTrP as is also explained on page 4, 1.3.3., by applying pressure to the muscle tissue by means of the “TRIGGOsan-Schlüssel”, which was an invention of the inventor.

This diagnostic method is very tedious and requires a lot of effort and practice. It has the risk of resulting in tissue damage and bruising.

Pain and dysfunction of the musculosceletal system is often a result of MTrPs. They can be diagnosed in superficial muscles by digital palpation, or mechanically with the help of the “TRIGGOsan-Schlüssel”. The MTrP can be identified under the electron microscope in a muscle biopsy. They impose as fusiform nodules of sarcomers which form the Trigger-Point-Complex (TPC) when they increase in numbers. The TPC can be palpated within a taut band of muscle fibers.

The muscle biopsy as a diagnostic method is very uncomfortable for the patient and requires a surgical procedure to obtain the specimen. The tissue needs to be processed and examined under the electron microscope.

Another method for the diagnosis of MTrPs is the electro diagnostic examination of the muscle by needle electromyography. There is an over activity of the endplate region as a sign of excessive spontaneous release of Acetylcholin resulting in a pathologic end plate noise (EPN). This form of diagnosis is painful for the patient and time consuming.

Another method for the diagnosis of MTrPs is the digital palpation of the TB within the muscle in which a swollen and exquisitely tender nodule needs to be located representing the TPC. This examination requires significant experience and is painful and time consuming. The major disadvantage of this digital palpation method is that the diagnosis is almost impossible in voluminous, or hardened muscles as well as in muscles which are covered by fat layers, intervening muscles and tissue.

Another well known method for diagnosing MTrPs is the so called “dry needling”. The MTrP is being identified by the elicitation of referral phenomena (RP) i.e. pain, numbness, tingling, heaviness or other sensory disturbances as well as the elicitation of the Local Twitch Response (LTR).—This method is difficult to learn, requires extensive experience, is time consuming and at times extremely painful for the patient.

Therefore it is an object of the invention to develop a diagnostic procedure for MTrPs which is simple to use, does not require much time and training and which causes minimal pain and minimal risk of tissue damage.

This can be achieved according to the invention by utilizing radial shockwaves for the diagnosis of MTrPs. Preferably these radial shockwaves have a higher energy density and/or a higher pressure generated by the shockwave. The diagnosis according to the invention is especially useful for finding referral phenomena (pain, numbness, tingling, heaviness and other sensory disturbances).

In a preferred embodiment the radial shockwaves have a maximum positive output pressure of about 30 Mpa upon entering the body.

The inventor found that the radial shockwaves which are being used for the therapy lend themselves for the diagnosis of MTrPs. But they require a significantly higher energy flux density than it is needed for the treatment of MTrPs.

An alternative especially preferred approach of the invention is the use of focused shockwaves for the diagnosis of MTrPs, especially to produce the RP and the LTR (see above).

In a preferred embodiment of the invention the focused shockwaves have an ED between 0.50 mJ/mm² and 2.0 mJ/mm².

In an especially preferred embodiment of the invention the focused shockwaves have a positive energy flux density of 0.78 mJ/mm².

The generation of focused shockwaves is explained in more detail—besides the already mentioned source—in the Internet publication of the “Medea Gesellschaft für alternative Behandlungsmethoden in der Medizin mbH under www.medea-online.com with the title “Physikalische Grundlagen der Extrakorporalen Stosswellentherapie” with the reference ©SGST/MEDEA GmbH (2001). From there it is known that focused shockwaves can be used for the treatment of calcifications, and the disintegration of kidney stones by means of focused shockwaves under the use of an appropriate reflector.

The use of focused shockwaves is as mentioned above known from the “Instruction Manual” for the device “PIEZOSON (100)” of the company. Richard Wolf, GA-A 174 /en/ Index: 01-03-4.0/ÄM: KG 03-009”, dated from 2001. This publication is also incorporated by reference.

The method according to the invention offers numerous advantages, among which:

It is simple to use, causes minimal pain and minimal risk of tissue damage. Especially MTrPs in deep tissue structures can be localized with focused shockwaves, which could not be reached otherwise or only with special effort. It can be utilized much more rapidly and thereby makes it possible to generate faster and more precise Trigger Charts. These are especially important to document the RP. Through the stimulation of a MTrP pain can be experienced not only locally but at a remote site of the body. This can be helpful in studying the characteristic RP of MTrPs as well as other tissues which have the potential of eliciting RP upon stimulation.

The above mentioned problems with the diagnosis can be solved through the invention by using shockwaves, in particular focused shockwaves, for the identification of MTrPs and other tissue areas from which RP can arise upon shockwave stimulation.

The so called LTR can be elicited with radial shockwaves of sufficient intensity and especially with focused shockwaves. This is an additional valuable tool for the diagnosis of MTrPs and membrane instability of synapses in general. This form of diagnosis does not require the patients feedback. Therefore it is especially useful for the diagnosis and treatment of neuro-muscular and musculosceletal problems of animals. The inventor has already achieved good results in applying this method to horses and dogs as a diagnostic and therapeutic tool.

The generation of shockwaves can be achieved by air pressure (ballistic shockwaves), piezoelectric, electrohydraulically, electromagnetically, Laser or by other means. The mode of generation is not relevant to the invention. For details regarding this issue special reference is being made to the above mentioned Internet publication of the MEDEA GmbH.

The focused shockwaves for diagnostic purposes are in the high- to middle-energy range. Low energy range means 0.08-0.12 mJ/mm², middle energy range means 0.12-0.28 mJ/mm² High energy range means>0.28 mJ/mm² until <2 mJ/mm². (see the mentioned Internet publication of the MEDEA GmbH).

The invention pertains also to a device to be utilized for the diagnosis of MTrPs. This is a device in principal like the Piezoson 100 of the company R. Wolf. For the use of radial shockwaves a device like the Swiss DolorClast by the company EMS, or Stortz Medical may be used.

Especially useful is the device OrthoWave 180 C of the company MTS Europe GmbH, Konstanz Germany. The parameters of this device are listed in the attached table.

TABLE 1

| | of the | | |
|---|---|---|---|
| OrthoWave 180 ® | company MTS | | |
| Shock wave | Minimum | Medium | Maximum |
| parameters | (step a) | (step 3) | (step 6) |
| Pos. peak | 2 | 30.7 | 31.4 |
| pressure/MPa | | | |
| Neg. peak | −1.5 | −4.2 | −7.2 |
| pressure/Mpa | | | |
| −6 dB-focal | 4.3 | 6.9 | 9.8 |
| extension x/mm | | | |
| −6 dB-focal | 4.3 | 6.9 | 9.8 |
| extension y/mm | | | |
| −6 dB-focal | 38 | 51.9 | 91.9 |
| extension z/mm | | | |
| −6 dB-focal | 480 | 1,293.8 | 4,621.3 |
| volume/mm³ | | | |
| energy flux | 0.004 | 0.148 | 0.331 |
| density (pos.)/ | | | |
| mJ/mm² | | | |
| energy flux | 0.005 | 0.155 | 0.357 |
| density (total)/ | | | |
| mJ/mm² | | | |
| energy −6 dB | 0.18 | 2.75 | 10.12 |
| (positive)/mJ | | | |
| energy −6 dB | 0.2 | 2.94 | 10.18 |
| (total)/mJ | | | |
| optional | | | |
| −6 dB-focal | 3.3 | 5.1 | 7.2 |
| extension x/mm | | | |
| −6 dB-focal | 3.3 | 5.1 | 7.2 |
| extension y/mm | | | |
| −6 dB-focal | 15 | 25 | 32 |
| extension z/mm | | | |
| energy flux | 0.01 | 0.34 | 0.52 |
| density (total)/ | | | |
| mJ/mm² | | | |
| energy −6 dB | 0.2 | 2.94 | 10.18 |
| (total)/mJ | | | |

According to the invention the frequency of the device can be chosen within a range of 4-5 Hz, the preferred frequency lies between 6-8 Hz. In the known device by the company R. Wolf a frequency of 4 Hz was the standard. The device of the company MTS goes up to a frequency of 5 Hz.

The depth of penetration which may be achieved varies between 0.1 cm and 10 cm.

For diagnostic purposes the preferred range of energy lies between 0.5-2.0 mJ/mm², and in a especially preferred case around 0.78 mJ/mm².

In summary a method and a device are provided, which in contrast to the methods used up to this point, do not cause injury to the skin, the subcutaneous tissue and fat, the connective tissue and the muscles. Furthermore the invention does not result in pain reactions which can last for several days or bruising and hematoma which could persist even much longer. MTrPs can be found which cannot be palpated easily i.e. MTrPs of the Mm. Multifidi and rotatores of the spine, the M. Gluteus medius and M. Gluteus minimus

### EXAMPLE

As an example the parameters of three different devices used for the diagnosis of MTrPs are listed in the table. One device was a piezoelectric shockwave emitter [PESE] [Piezoson 100] with a total energy [ED] of 0.262-1.64 mJ/mm², a −6 db focal volume [−6 db FV] of 58.8-96.1 mm3 and a focal extension [z] of 8.2-11.7 mm. The other device was an electro-hydraulic shockwave emitter [EHSE] [Orthowave 180 C] with ED 0.005-0.357 mJ/mm², a −6 db FV of 480-4621.3 mm3 and z=38.0-91.9 mm. The shockwaves were applied with the ED adjusted to a pain level of 60 on the visual analog scale [VAS].

114 cases with chronic sciatica type pain, unresponsive to conventional treatments were examined for the presence of active MTrPs in the gluteus medius and minimus muscles. 57 cases with a piezoelectric shockwave emitter [PESE] with a total energy [ED] of 0.262-1.64 mJ/mm², a −6 db focal volume [−6 db FV] of 58.8-96.1 mm3 and a focal extension [z] of 8.2-11.7 mm. 57 cases with an electro-hydraulic shockwave emitter [EHSE] with ED 0.005-0.357 mJ/mm2, a −6 db FV of 480-4,621.3 mm3 and z=38.0-91.9 mm. The shockwaves were applied with the ED adjusted to a pain level of 60 on the visual analog scale [VAS]. Results: The diagnosis of active MTrPs with PESE/EHSE required an ED of 0.82/0.266 mJ/mm2. The familiar referred pain, indicative of active MTrPs were obtained in all cases. Conclusions: Focused shockwaves can be used to diagnose active MTrPs in muscles which are not accessible to palpation. PESE requires a 3 times higher ED, causes a higher local pain versus no or dull pain with the EHSE. These effects can be explained by its up to 50 times smaller −6 db FV compared to the EHSE −6 db FV. This approach allows to continuously diagnose during the treatment and to validate it against the treatment outcome.

The average values of this large clinical trial are listed in the table. The depths of penetration was measured using a diagnostic ultrasound device. It became apparent that the pain sensation during diagnosis was significantly less using the EHSE compared to the PESE. This could be explained by the wider focus area of the EHSE and a resultant lower energy flux density of 0.266 mJ/mm² compared to 0.82 mJ/mm² with the PESE.

TABLE 2

| | Minimum | | | Medium | | | Maximum | | |
|---|---|---|---|---|---|---|---|---|---|
| | | EHSE | | | EHSE | | | EHSE | |
| Shock | PESE | ELLIPSOID | EHSE | PESE. | ELLIPSOID | EHSE | PESE | ELLIPSOID | EHSE |
| wave | Reflector | “0.52 | ELLIPSOID | Reflector | <<0.52 | ELLIPSOID | Reflector | “0.52 | ELLIPSOID |
| parameters | 12 cm | mJ/mm²>> | <<0.35 mJ/mm²>> | 12 cm | mJ/mm²>> | <<0.35 mJ/mm²>> | 12 cm. | mJ/mm²” | “0.35 mJ/mm²” |
| Pos. peak | 24.7 | 2.0 | 5.0 | 75.1 | 30.7 | 37.2 | 122.5 | 31.4 | 51.0 |
| pressure | | | | | | | | | |
| [Mpa] | | | | | | | | | |
| Tensile | 0.139 | 0 | 0 | 0.31 | | 0.004 | 70 | <10 | 0.025 |
| wave | | | | | | | | | |
| [% of | | | | | | | | | |
| peak] | | | | | | | | | |
| −6 db | 3.1 | 4.3 | 3.7 | 1.4 | 6.9 | 4.7 | 1.4 | 9.8 | 5.9 |
| focal | | | | | | | | | |
| extension | | | | | | | | | |
| x [mm] | | | | | | | | | |
| −6 db | 3.1 | 4.3 | 3.8 | 12.0 | 6.9 | 4.8 | 16.0 | 9.8 | 5.9 |
| focal | | | | | | | | | |
| extension | | | | | | | | | |
| y [mm] | | | | | | | | | |
| −6 db | 11.7 | 38.0 | 18.7 | 6.5 | 51.9 | 31.3 | 8.2 | 91.9 | 35.3 |
| focal | | | | | | | | | |
| extension | | | | | | | | | |
| z [mm] | | | | | | | | | |
| −6 db | 58.84 | 480.0 | 289.3 | 57.15 | 1,293.8 | 729.9 | 96.13 | 4,621.3 | 650.3 |
| focal | | | | | | | | | |
| volume | | | | | | | | | |
| [mm³] | | | | | | | | | |
| Energy | 0.123 | 0.004 | 0.01 | 0.48 | 0.148 | 0.266 | 1.08 | 0.331 | 0.495 |
| flux | | | | | | | | | |
| density | | | | | | | | | |
| (positive) | | | | | | | | | |
| [mJ/mm²] | | | | | | | | | |
| Energy | 0.262 | 0.005 | 0.01 | 0.79 | 0.155 | 0.270 | 1.64 | 0.357 | 0.520 |
| flux | | | | | | | | | |
| density | | | | | | | | | |
| (total) | | | | | | | | | |
| [mJ/mm²] | | | | | | | | | |
| Energy − | 0.79 | 0.18 | 0.35 | 0.61 | 2.75 | 6.07 | 1.42 | 10.12 | 9.20 |
| 6 db | | | | | | | | | |
| (positive) | | | | | | | | | |
| [mJ] | | | | | | | | | |
| Energy − | 1.63 | 0.2 | 0.37 | 0.97 | 2.94 | 6.15 | 2.62 | 10.18 | 9.38 |
| 6 db | | | | | | | | | |
| (total) | | | | | | | | | |
| [mJ] | | | | | | | | | |

1. A method for treating myofascial trigger points and/or other tissue comprising applying focussed shockwaves to a patient in need thereof.

2. The method of claim 1 wherein the focussed shockwaves have a total flux density between 0.10 mJ/mm² and 3.0 mJ/mm².

3. A method for diagnosing myofascial trigger points comprising applying radial shockwaves to a patient.

4. The method of claim 3 wherein the myofascial trigger points are ones that cause referral phenonema, such as pain, numbness, tingling, heaviness, and other sensory disturbances upon stimulation with shockwaves.

5. The method of claim 3 wherein the radial shockwaves have a maximum positive output pressure of 30 MPa upon entering the patient's body.

6. The method of claim 4 wherein the radial shockwaves have a maximum positive output pressure of 30 MPa upon entering the patient's body.

7. A method for diagnosing myofascial trigger points comprising applying focussed shockwaves to a patient.

8. The method of claim 7 wherein the myofascial trigger points are ones that cause referral phenomena such as pain, numbness, tingling, heaviness, and other sensory disturbances upon stimulation with shockwaves.

9. The method of claim 7 wherein the focussed shockwaves have a total energy flux density between 0.50 mJ/mm² and 2.0 mJ/mm².

10. The method of claim 7 wherein the focussed shockwaves have a positive energy flux density of 0.78 mJ/mm².

11. A shockwave emitter for the diagnosis of myofascial trigger points using focussed shockwaves comprising means for emitting shockwaves at a rate between 5 and 8 Hz.
